(19) United States
(12) Patent Application Publication
(10) Pub. No.: US 2015/0265380 A1
(43) Pub. Date: Sep. 24, 2015

(54) COMPANION ENGINEERING AND MANUFACTURING PROCESSES (CEMP) TO OPTIMIZE MULTI-LAYERED ZIRCONIA CROWNS

(71) Applicant: ZIRCORE, LLC, Manhattan, KS (US)

(72) Inventors: ROBERT DAVID SAGER, MANHATTAN, KS (US); Peter R. Hunt, Philladelphia, PA (US); Bernard Keough, Palm Beach Gardens, FL (US); Howard Kay, West Palm Beach, FL (US); Eric Keen, Manhattan, KS (US)

(21) Appl. No.: 14/579,647

(22) Filed: Dec. 22, 2014

Related U.S. Application Data
(63) Continuation of application No. 13/775,910, filed on Feb. 25, 2013.
Application No. 13/775,910 is a continuation of application No. 14/300,856, filed on Jun. 10, 2014.
Application No. 14/300,856 is a division of application No. 12/346,341, filed on Dec. 30, 2008, now Pat. No. 8,751,031.
Application No. 12/346,341 is a continuation-in-part of application No. 11/107,519, filed on Apr. 15, 2005, now Pat. No. 7,690,920.
Application No. 11/107,519 is a continuation-in-part of application No. 12/212,256, filed on Sep. 17, 2008, now Pat. No. 7,967,606.
Application No. 12/212,256 is a continuation of application No. 11/023,950, filed on Dec. 28, 2004, now Pat. No. 7,445,449.
(60) Provisional application No. 61/602,571, filed on Feb. 23, 2012.
(60) Provisional application No. 61/099,566, filed on Sep. 24, 2008.
(60) Provisional application No. 60/631,102, filed on Nov. 26, 2004.
(60) Provisional application No. 60/566,855, filed on Apr. 30, 2004.
(60) Provisional application No. 60/543,038, filed on Feb. 6, 2004.

(51) Int. Cl. A61C 13/00; A61C 13/08; A61K 6/02; A61C 8/00; A61C 5/10

(52) U.S. Cl. CPC A61C 13/0004; A61C 8/0086; A61C 5/10; A61C 13/0006; A61K 6/024; A61C 13/082

(57) ABSTRACT

A companion engineering and manufacturing process to optimize multi-layered zirconia crowns, and improved substructure are provided.

### CROSS-REFERENCE TO RELATED APPLICATIONS

This application is a continuation of U.S. application Ser. No. 13/775,910, filed on Feb. 25, 2013, which claims priority pursuant to 35 U.S.C. 119(e) to co-pending U.S. Provisional Patent Application Ser. No. 61/602,571, and also is a continuation of U.S. application Ser. No. 14/300,856 filed on Jun. 10, 2014, which is a divisional application of then co-pending U.S. application Ser. No. 12/346,341 filed on Dec. 30, 2008 (now U.S. Pat. No. 8,751,031) and which claims priority pursuant to 35 U.S.C. 119(e) to then co-pending U.S. Provisional Patent Application Ser. No. 61/099,566, filed Sep. 24, 2008, and which is a continuation-in-part application of U.S. application Ser. No. 11/107,519 filed Apr. 15, 2005 (now U.S. Pat. No. 7,690,920) and of U.S. application Ser. No. 12/212,256 filed Sep. 17, 2008 (now U.S. Pat. No. 7,967,606), which is a continuation of U.S. application Ser. No. 11/023,950 filed Dec. 28, 2004 (now U.S. Pat. No. 7,445,449), which claims priority pursuant to 35 U.S.C. 119(e) to then co-pending U.S. Provisional Patent Application Ser. No. 60/631,102, filed Nov. 26, 2004, U.S. Provisional Patent Application Ser. No. 60/566,855, filed Apr. 30, 2004 and U.S. Provisional Patent Application Ser. No. 60/543,038, filed Feb. 6, 2004, the entire disclosures of which are incorporated herein by reference.

### FIELD OF THE INVENTION

The present invention relates generally to prosthodontic systems, methods and apparatuses. More particularly, the present invention is concerned with crowns and bridges and a process for manufacturing dental crowns and bridges. Even more particularly, the present invention is concerned with improved embodiments of, and improved methods/processes of manufacturing crowns and bridges that utilize a high strength substructure reinforcement for crowns and bridges, such as is disclosed in U.S. Pat. No. 7,690,920, the entire disclosure of which is incorporated herein by reference.

### BACKGROUND OF THE INVENTION

For some time, dental prostheses were produced from porcelain veneering material bonded onto a metal framework (substructure, or core). This porcelain-fused-to-metal (“ceramo metal”) construction required a fairly bulbous metal core, with a fairly uniform porcelain thickness to avoid de-lamination under sheer conditions. More recently, dentists have been offered high strength ceramic materials as substitutes for the conventionally metal substructures in ceramo-metal crowns and/or bridges. These porcelain veneer fused to non-metal core crowns/bridges are expected to offer equivalent or superior precision to ceramo-metal alternatives.

Particular emphasis has recently centered in the strength of the various new non-metal substructure materials. Traditional ceramic cores have compressive strengths in the region of 150-200 MPa. Alumina based cores claim strengths in the region of 400-600 MPa, and Zirconia cores have strengths ranging from 900-1200 MPa. Notwithstanding the impressive strengths of these substructure materials, these figures are deceptive due to the more limited strength of the veneering materials used for the crowns and/or bridges. Weaker veneering materials if not adequately supported by the higher strength ceramic core, often result in fracturing of the veneering porcelain while the ceramic core remains intact.

In restorations supported by implants instead of teeth, the fracture potential may be increased even more. This increased fracture potential may be caused in part by redistribution of bulks of veneering porcelain. First, implants tend to be placed more lingually than the teeth they are replacing, which often results in more unsupported buccal porcelain. Second, the diameter of an implant platform is often smaller than the tooth it is replacing, giving rise to opportunity for still bigger bulks of unsupported porcelain. The situation is further complicated by implants being more rigid than teeth, while at the same time inherently reducing proprioceptive capabilities. All these factors tend to concentrate and accentuate the forces placed on the veneering porcelain.

Fracture of veneers is not a new problem. Traditionally, in ceramo-metal technology, the weakness of the veneering ceramic material has been compensated for by bulking up and designing the substructure in certain quite well defined ways. Nevertheless, bulking up the substructure often results in a less desirable aesthetic appearance, as the substructure becomes more visible through the veneer. One example of an attempt to provide a concealed support includes constructing a metal collar at the base of the substructure with a shoulder brought part way up the lingual surface of the substructure. Also, in the interproximal regions the substructure frame is frequently built out under the contact points with the veneer. The concept behind these design elements is to reduce the bulk of the veneering porcelain and to convert the loading stresses on the veneer ceramic from being in shear to being under compression.

Many operators are applying the same concepts traditionally used for ceramo-metal technology to the design of high strength ceramic substructures. Notwithstanding, these design elements are not much help in strengthening the most visible portions of the teeth, the buccal surfaces. This can be a particular problem in the mandible because in a normally related occlusion, the buccal cusps of the mandibular teeth serve as occlusal supports. Using a high strength ceramic substructure, which tends to be opaque and of high value, half way up the buccal surface to reinforce the veneer porcelain of the cusp is just as unacceptable as using a metal frame in the same manner. Furthermore, a marginal collar at the interproximal regions of the substructure provides little or no reinforcement because it is so far away from the region where the stress is being applied (i.e. the tip of the buccal cusps).

The invention disclosed in U.S. Pat. No. 7,690,920 provides a significant strengthening mechanism for a crown and/or bridge close to the region where the stress is being applied to the veneer that does not compromise aesthetics and which is relatively simple to design and construct. Notwithstanding, it is desirable to provide engineering and manufacturing processes that optimize such multi-layered restorations.

Furthermore, dental crowns and bridges have evolved from all metal to ceramic fused to metal and all ceramic, porcelain fused to metal and all ceramic crowns and bridges of the prior art, and all are primarily a result of an artisan or technology enhanced artisan fabrication process often requiring multiple processes and material layering. Prior art typically employed an opaquer layer to mask the darkness/color of metal cores or modify the transparency/translucency of ceramic cores. To date, the opaque process has been a hand-eye coordinated process involving the application of various finely milled/precipitated powders mixed with some fluid vehicle, either by hand or hand held spray unit. At all times, an artisan eye process has been employed to develop color and character even if color, chroma, hue, values, etc. were first obtained by digital scan methods of prior art. To date, an intermediary color/character conversion method has been employed to remove all that is digital to analog and then relying on human judgment to decide how to achieve an artisan result. In art from antiquity, a trompe l'oeil technique has been employed to trick the eye into seeing a desired result, regardless of the base condition at hand. It is desirable to provide a process that eliminates or reduces the manual hand-eye coordination to obtain the desired color and character, chroma, hue, etc.

### SUMMARY OF THE INVENTION

An object of the present invention is to provide a process and improved process for manufacturing multilayer prosthodontic devices. Another object of the instant invention is to provide a process and improved process for manufacturing prosthodontic devices having a high strength/reinforced substructure. Another object of the instant invention is to provide improved prosthodontics device structures. Yet another object of the instant invention is to provide improved prosthodontic devices resulting from the improved manufacturing process in addition to enhanced material science and engineering. Still another object of the instant invention is to provide improved prosthodontic devices resulting from the improved manufacturing process in addition to improved colorization or color correction/value, or modification/optimization of aesthetics of/to the whole device.

The objects of the instant invention are achieved through engineering and manufacturing processes (“EMP”) that use prosthodontic substructures that include an annular reinforcement structure generally running around the center of the crown of the substructure, such as is disclosed in U.S. Pat. No. 7,690,920 (a/k/a “Buttress Ring™”, “BR”, and/or “ring beam”). It will be appreciated that various embodiments of the instant invention utilize one or more components and/or subcomponents of the inventive systems, methods, apparatuses and/or processes disclosed in any of U.S. application Ser. No. 12/346,341, filed Dec. 30, 2008, U.S. Provisional Patent Application Ser. No. 61/099,566, filed Sep. 24, 2008, U.S. application Ser. No. 11/107,519 (now U.S. Pat. No. 7,690,920), filed Apr. 15, 2005, U.S. application Ser. No. 12/212,256 (now U.S. Pat. No. 7,967,606), filed Sep. 17, 2008, U.S. application Ser. No. 11/023,950, filed Dec. 28, 2004 (now U.S. Pat. No. 7,445,449), U.S. Provisional Patent Application Ser. No. 60/631,102, filed Nov. 26, 2004, U.S. Provisional Patent Application Ser. No. 60/566,855, filed Apr. 30, 2004 and U.S. Provisional Patent Application Ser. No. 60/543,038, filed Feb. 6, 2004 (the entire disclosures of which are incorporated herein by reference), either alone or in combination with components and/or subcomponents of one another and/or other components and/or subcomponents now known or hereinafter discovered.

Embodiments of the instant invention are discussed in detail in the article titled “Clinical Performance of Scientifically Designed, Hot Isostatic-Pressed (HIP'd) Zirconia Cores in Bilayered All-Ceramic System”¹, the entire disclosure of which is incorporated herein by reference. In one embodiment of the invention, the engineering and manufacturing process includes the following steps:
1. The structure includes an optimized sintered zirconia core: End state isotropic HIP'd (Hot Isostatic Pressed) zirconia.
2. The zirconia Core contains a Buttress Ring™, “CBR”—an annular reinforcement ring beam structure that rings the radius of the Core, such as is disclosed in U.S. Pat. No. 7,690,920.
3. The CBR is veneered on the outer generally convex surface with porcelain or other material with an ideal thermal expansion coefficient that is generally ≦1.0×10⁻⁶/° C. of CBR material.
4. The first layer of porcelain applied to the CBR is a modifier/transition layer, that exists between the body of porcelain and the CBR.
5. The modifier/transition layer and successive layers of porcelain are fired at an “ideal temperature” that is preferably slightly less than the veneer manufacturer's recommended high firing temperature and then withdrawn in a special rapid cool-down protocol.
6. The modifier/transition layer and successive layers of porcelain may also be applied in a molten/phase-change state such as a plasma spray application in an “ideal application” process.
7. In all cases, rapid controlled, thermodynamic principles are followed to reduce transient heat transfer between materials, which may be dissimilar to traditional or ceramo-metal protocols. Keough, D M D Bernard E. et al. “Clinical Performance of Scientifically Designed, Hot Isostatic-Pressed (HIP'd) Zirconia Cores in Bilayered All-Ceramic System.” Compendium of Continuing Education in Dentistry: North American Edition. July/August 2011, Volume 32, Number 6: 58-68.

Buttress Rings™ provide optimum Core geometry for elimination of function induced tensile stresses on the veneering porcelain through elimination of core flexure, occlusal support of the veneering porcelain, and increased surface area for mechanical retention of porcelain onto zirconia CBR. In some embodiments, an occlusal/superior-circular/coronal ring is provided generally below the cusps and along the marginal ridges. This second ring acts to “zone/subdivide” the veneering porcelain to increase the strength of veneering bond by “design.” A “fish mouth” style of some embodiments of this occlusal ring also allow the reduction of mass of the core in a non-critical area of Core strength. This affords increased esthetics (i.e. through thicker porcelain). It also improves porcelain veneering by “regularizing” and/or “standardizing” the morphology of similar pieces of different patient cases such that the thermodynamics can be more closely industrially controlled. The purpose of the two rings (Buttress™ and Occlusal) is to make the greatest thickness of veneering porcelain, that will take longer to cool/shrink and consequently contribute the highest “compression” to best fixate the porcelain to the walls. The rings act as a physical “tie” for the porcelain to the core/substructure. In another way, in the world of ceramic compression, the rings act as a “corrugation” of the surface that affords increased surface area and increased zones of compression, that can be ideally positioned for the desired result of better retaining veneering porcelain and to reduce localized “chipping and spalling.”

The Modifier/Transition Layer provides increased veneering bond strength and veneer integrity. Embodiments of this layer include, but are not necessarily limited to the following:
1. In one embodiment, the layer serves as a companion-engineered nano-bond layer that enhances the bond of veneering porcelain via ideal/special particle size and/or particle charge or other scientific principle now known or hereinafter discovered.
2. In another embodiment, the layer serves as a trompe l'oeil special effect-shading layer that enhances the esthetic appearance of the porcelain via ideal/special particle composition and/or crystal structure or other scientific principle now known or hereinafter discovered.
3. In one other embodiment, the layer is CAD/CAM 3D printed to the surface of the Core to facilitate porcelain veneering of Cores. It will be appreciated that such “printing” may be accomplished via 3D plasma spraying, 3D thermal printing, or any other computer aided application of porcelain to HIP'd Zirconia now known or hereinafter discovered that does not require the Core temperature to rise to the temperature of the porcelain's “molten” state however it is applied to the Core.

Optimum companion TEC provides optimum compressive forces of veneer surface at interface between CBR and veneering porcelain.

The rapid cool down protocol provides optimum compression and increased strength on the external surface of the veneering porcelain.

Companion EMP (“CEMP”) minimize stress fractures and delamination of porcelain from GBR and fracture and chipping from within veneer porcelain layer.

The instant invention utilizes the inherent heat refraction properties and isotropic/HIP'd form of Zirconia (or other suitable materials hereinafter discovered) to result in an improved bond between the Core and the porcelain layer. The instant invention favors all physical qualities of the bond and resulting mathematics related to the bond and veneer to become a matter of simplified linear calculations. The inclusion of the CBR in the instant invention provides increased surface area for “adhesion” of the porcelain layer, and increased thickness of the Core to further increase the “R” (heat resistance/refraction) value of the material to resist temperature changes, to provide a unique and improved bond between the Core and porcelain layer.

It will be appreciated that the porcelain layer may comprise natural or synthetic materials now known or hereinafter discovered, including but not limited to such natural materials as feldspathic or silicon, and such synthetic materials as Lucite or other crystal formulations.

In one preferred embodiment of the instant invention, the substructure (core) is made of a non-metal material. Instead of merely constructing a thin core 20 over the foundation 10 (as is shown in FIG. 1 of U.S. Pat. No. 7,690,920), and instead of attempting to replicate the design structure used for a porcelain fused to metal restoration of the prior art, the reinforcement structure is designed at or about the height of contour of the crown. This is where the greatest thickness of veneering porcelain is usually located and lies right under where occlusal stresses will be applied.

In one preferred embodiment, the reinforcement structure is incorporated into the CAD-CAM design stage for a high-strength, milled substructure. The design of this reinforcement structure depends on the software associated with each CAD-CAM system. The reinforcement structure may be designed primarily manually using conventional CAD-CAM design software, which allows a user to place pre-made shapes down over an image on the screen, increase or decrease the existing shape, distort the shape in one/multiple vanishing points, increase the volume of the shape from a point-angle or free hand-paint an area. In one preferred embodiment, the reinforcement structure is designed primarily automatically by a CAD-CAM (or other software) application that includes a pull down annular shape that is placed around the concentric image being made over the die from a library of tools in the software palate. The annular shape may be enlarged (or reduced) in x, y, and z axis as necessary to bulk-up (or down) the core being designed. In another preferred embodiment, the reinforcement structure is designed primarily automatically by a CAD-CAM (or other software) application by the operator pulling the structure from a point, line, cluster of points, etc., to distort a portion of the shape of the main body of the substructure to create the reinforcement structure without distorting the overall shape of the main body. In yet another preferred embodiment, the reinforcement structure is pre-designed into the main body of the substructure. In such an embodiment, a basic shape for the main body of the substructure is selected from a library of shapes available in the software application based upon the desired shape for the final restoration, with the reinforcement structure already built into the shape of the main body. The operator then either pushes, pulls, takes away or otherwise erases portions of the pre-designed shape of the main body (including the reinforcement structure) to meet the needs for the specific restoration. In still another preferred embodiment, the substructure (including the reinforcement structure) is designed by first obtaining the desired shape for the restoration and then subtracting away or deconstructing from that shape to leave the substructure shape. In such an embodiment, the part of the final shape that is subtracted is determined to maximize the aesthetic appearance of the final restoration by concealing the substructure. One such embodiment of designing the substructure through deconstruction, the system and method of U.S. Pat. No. 7,967,606 is utilized in combination with the structure and methods of the instant invention discussed herein.

In one embodiment the reinforcement structure is over-built in the CAD-CAM design phase on a relatively freehand basis (or through use of the automatic software discussed above), preferably at the crest of the preparation, and protruding about 2 mm out from the base core. After milling is completed, the contact regions are adjusted, and the amount of the lingual and labial prominence is modified as desired, by hand. If necessary or desired, the reinforcement structure is thinned out after milling. There is little requirement for bulk of the reinforcement structure because the high-strength non-metal substructure is not only strong, it is also very rigid. The final frame design is easy to design, construct and manage.

One embodiment utilizes the unique material capabilities of Hip'd zirconia or other similar material not yet discovered to foster direct application of sequentially applied porcelain or other compatible veneering materials, whether CAD/CAM applied via 3D printing(s) in a dry state followed by heat application(s) or via progressive “molten”/phase-changed material applications.

The aesthetic appearance of the piece is increased by reducing the potential for the sub-structure to “Shine-Through” the surface of the final restoration at the mesio-buccal region. To minimize Shine-Through, the prominent sub-structure may be reduced through the crown contour. Veneering porcelain is then applied over the deficiency. In addition, the reinforcement structure may be masked when the final restorations are characterized.

The reinforcement structure has the advantage that the marginal display of opaque porcelain from the underlying high strength core can be minimized, because there is no need for a heavy bulky collar to gain strength or to provide support. The normal thickness of the base core can be extended to the margin. This is particularly useful in implant based units, where there tends to be a bigger build out from a relatively narrow base.

Although the scope of the instant invention is not limited to any specific materials for the substructures (or the veneers), it will be appreciated that a reinforcement structure is particularly well-suited for use with non-metal substructures. Due to the differential in thermal coefficients of expansion for most metal substructures from that of the overlying porcelain, a metal framework would tend to cool faster than the ceramic, possibly resulting in cracks in the veneer porcelain. In addition a more complex ceramic veneer construction may be needed to mask out a metal substructure than is necessary for a high strength ceramic framework.

The reinforcement support is remarkably simple to incorporate and use in practice. In one embodiment of the instant invention, for single unit restorations, a thin layer core is designed over the preparation (such as a pre-manufactured implant abutment, a custom manufactured implant abutment, prepared portion of tooth on which restorations is supported, etc.) with a relatively crude shaping of the reinforcement structure. In a preferred embodiment, the reinforcement structure is established at the height of the contact point and parallels the occlusal plane. When the reinforcement structure has been established relatively crudely in the design stage, it can then be refined quickly once the unit is positioned on a master model.

In some embodiments of the instant invention, the Modifier/Transition layer is utilized to impart colorization/color correction/value, or modification/optimization of aesthectics of/to the whole piece. In some such embodiments, a Custom automated mass manufacturing CAM²™ process is utilized which requires a translation of all that is known in nature and science into a digital ordering and sequencing format of algorithmic that can be employed in a preplanned or instantaneous manner to drive technology to provide a desired product. In such embodiments of the instant invention information is utilized from a central protect file and reference libraries to layer layers of materials intended to serve as zones of colorization, characterization and bonding within the multi layer ceramo metal or all ceramic crowns and bridges. While the process and results may be modified by eye, it is the intention of the instant invention for the process to be highly automated so as to not require the human eye and mind to control the process of an individual unit basis. Embodiments of the instant invention employ techniques and technologies of printing and painting from prior art, but at all times repurpose them to the CAM process for dental crowns and bridges. The instant invention applies materials at the inner core layer (i.e. modifier/transition layer) of ceramic fused to metal crowns or multi layer all ceramic crowns to enhance the bond of successive ceramic layers and/or color and characterize the base layer and/or successive ceramic layers including the outer most final contour layer of the finished crown or bridge.

Some embodiments of the instant invention utilize the mass custom manufacturing system and methods disclosed in U.S. application Ser. No. 12/346,341 in combination with the structures and methods of the instant invention discussed herein. In some such embodiments the deconstruction design method of U.S. Pat. No. 7,967,606 is also utilized as part of the combination.

Some embodiments of the instant invention utilize the continuous production method of U.S. Pat. No. 7,445,449 in combination with the structures and methods of the instant invention discussed herein. In some such embodiments the deconstruction design method of U.S. Pat. No. 7,967,606 is also utilized as part of the combination. In other embodiments, the mass custom manufacturing system and methods disclosed in U.S. application Ser. No. 12/346,341 is utilized as part of the combination (in some embodiments with the methods of U.S. Pat. No. 7,967,606, and in some embodiments without).

Some embodiments of the instant invention apply materials in such a way as to impart new color and character to and/or mask color and character from the treating surface. The materials may in single layer effect or multiple layering effect develop a trompe l'oeil quality and achieve the most life like result with out of body materials and techniques.

Some embodiments of the instant invention employ modern inkjet printing and charged particle coating technologies of prior art to achieve the desired technological and esthetic results. Nevertheless, it will be appreciated that other coating technologies now known or hereinafter developed may be utilized without departing from the spirit and scope of the instant invention.

In some embodiments, the colorization layer is applied to the outside of the piece (porcelain buildup) in a coordinated manner to enhance the final composite aesthetic result. In other embodiments the colorization layer is applied exclusively to the outside of the piece in the case of a monolithic crown or finished multi-layer crown.

In some embodiments, the color/characterization information for a piece is obtained from a remote or on-site electronic transmission from a digital imaging color “characteristic capture” system that scans the tooth being replaced or surrounding teeth of the patient.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

A preferred embodiment of the invention, illustrative of the best mode in which the applicant has contemplated applying the principles, is set forth in the following description and is shown in the drawings and is particularly and distinctly pointed out and set forth in the appended claims.

FIG. 1 is front elevation section view of a dental restoration including a conventional substructure.

FIG. 2 is a front elevation section view of a dental restoration of an embodiment of the instant invention.

FIG. 3 is a top plan section view of the dental restoration of FIG. 2

FIG. 4 is a front elevation section view of a dental restoration substructure of an embodiment of the instant invention.

FIG. 5a is a proximal section view of a dental restoration of another embodiment of the instant invention showing the areas of greatest compression of veneering porcelain.

FIG. 5b is an occlusal section view of the dental restoration of FIG. 5a.

### DETAILED DESCRIPTION OF A PREFERRED EMBODIMENT

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to FIG. 1, a dental restoration including a conventional substructure is shown. As is shown in FIG. 1, the restoration includes abutment portion 10 over which thin core 20 is fit. Crown 30 is formed about core 20 from a veneer porcelain to the shape of the final restoration. As can be seen in FIG. 1, a considerable amount of bulking out of the veneer porcelain is required to establish the final form of the restoration. The stresses from the mandibular posteriors tend radiate out from tip 35 of the buccal cusps. The conventional reinforcement structures, such as collar 12 located at the base of the substructure, are greatly limited in location due to aesthetic concerns. Because such reinforcement structures are located away from the region of stress, 35, fractures in the porcelain for crown 30 can occur.

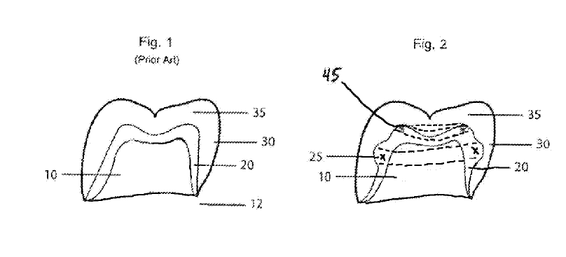

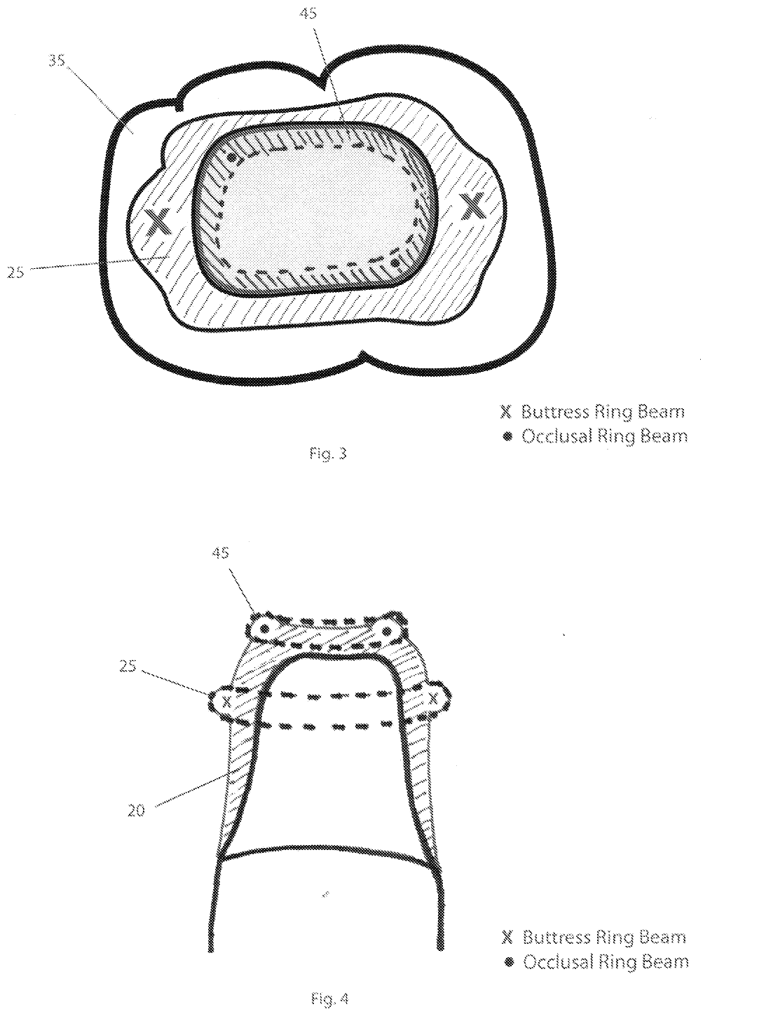

Referring to FIGS. 2 and 3, a dental restoration including an embodiment of the reinforced substructure of the instant invention is shown. As is shown in FIG. 2, the restoration includes abutment portion 10 over which core 20 is fit. Core 20 includes a body with a generally uniform outer surface and including annular protrusion 25 located generally near an occlusal portion of the restoration, and circular/superior protrusion (“Occlusal Beam” or “Coronal Beam”) 45 that generally runs below the cusps and along the marginal ridges. Crown 30 is formed about core 20 from a veneer porcelain to the shape of the final restoration in the manner discussed above. As can be seen in FIG. 2, protrusion 25 is located near the location of the most considerable amount of bulking out of the veneer porcelain that is used to establish the final form of the restoration. As with the conventional restoration shown in FIG. 1, the stresses from the mandibular posteriors tend to radiate out from tip 35 of the buccal cusps; however, protrusion 25 is located in close proximity to (right under in the shown embodiment) this high stress region to provide a contact region and support for the veneering porcelain of crown 30. In addition to providing support, locating the relatively thin reinforcement 25 in the region of the thickest veneering porcelain also allows protrusion 25 to be concealed by the porcelain. In addition, the location of reinforcement protrusion 25 allows core 20 to be relatively thin at the margin (base) resulting in optimal aesthetics for the restoration. The circular protrusion 45 acts to “zone/subdivide” the veneering porcelain to increase the strength of veneering bond by “design.” The “fish mouth” style of the embodiment shown in FIGS. 2 and 3 of this Occlusal Beam also allows the reduction of mass of the core in a non-critical area of Core strength. This affords increased esthetics (i.e. through thicker porcelain). It also improves porcelain veneering by “regularizing” and/or “standardizing” the morphology of similar pieces of different patient cases such that the thermodynamics can be more closely industrially controlled. The purpose of the two rings (Buttress™ 25 and Occlusal 45) is to make the greatest thickness of veneering porcelain, that will take longer to cool/shrink and consequently contribute the highest “compression” to best fixate the porcelain to the walls. The rings act as a physical “tie” for the porcelain to the core/substructure 20. In another way, in the world of ceramic compression, the rings act as a “corrugation” of the surface that affords increased surface area and increased zones of compression, that can be ideally positioned for the desired result of better retaining veneering porcelain and to reduce localized “chipping and spalling.”

It will be appreciated that the use of the phrase “generally uniform” to describe the outer surface of the body of core 20 of the instant invention refers to the fact that the outer surface of core 20 generally (other than protrusions 25 and 45) does not include any significant variations in contour from those variations typically found in conventional core structures which generally follow the contours of (i.e. are concentric with) the surface of the restorations for which they are substructures. Such contours often include various asymmetrical and irregular shapes that may include both concave and convex patterns in a single structure. Therefore, the generally uniform surface does not require the surface to be smooth or even, or of any standardized or symmetrical shape or form. It will further be appreciated that reference to the outer surface of the body of core 20 as being generally uniform is not intended to require a uniform thickness for core 20, even though the embodiment of core 20 shown in FIGS. 2 and 3 does include a generally uniform thickness.

As the shape of the outer surface of core 20 will vary significantly, so too will the shape of protrusions 25 and/or 45. Although shown and described in the preferred embodiment as a generally annular, symmetrical shape (as protrusion 25), it will be appreciated that the contact region/support structure (“protrusions”) of the instant invention may take on any number of shapes both symmetrical and asymmetrical, can be a single structure that generally encircles the substructure body (as is shown and described herein with respect to annular protrusion 25 and/or circular protrusion 45), can be multiple structures that generally encircle the substructure body, can be one or more substructures that partially encircle the substructure body, or can be one or more substructures that each protrudes from a single point along the substructure body. Furthermore, the terms “protrusion” and “protruding” are intended to include, but not be limited to, any convex shape that is not a coincident concentric duplicate shape of the preparation; any complex amplification of shape that is not just a convex derivative of the shape of the preparation; any shape having a concave approach from each side approach to an amplified area of protrusion that can not be described in simple harmonics, but only complex wave form; any superseding amplification of form that is not accidental or rendered for strictly artistic purpose; any concentric enlargement that is disproportionately distributed toward the superior (non-apical) portion of the long axis of the preparation; any asymmetrical appendage added to a design by computer generated pre-made shape or file added for specific structural considerations of subsequent materials added to a restoration infrastructure. Furthermore, it will be appreciated that the “protrusions” of the instant invention may be designed as an integral portion of an infrastructure (as is shown and described herein with respect to core 20 and protrusions 25 and 45), or alternatively, the “protrusions” may be one or more separate components that are attached to or otherwise combined with an infrastructure. Examples of “protrusion” shapes of the instant invention in addition to the generally annular protrusion 25 and circular protrusion 45 shown herein include but are not limited to bulges, power-swells, tumors, bumps, blobs, raised protuberances, etc.

As is shown in FIG. 1, conventional substructures tend to taper upward from the base of the substructure (located generally at the gums), so that the greatest thickness of veneering ceramic will be applied toward the top of the substructure, concealing the substructure at the location in which it would be most visible once finally installed. As is shown in FIG. 2, core 20 of the instant invention also includes this upward tapering shape. Thus, as is already mentioned above, it will be appreciated that the phrase “generally uniform” with respect to the outer surface of core 20 is not intended to limit the shape of the outer surface of core 20, which can be generally cylindrical, conical, generally convex (especially for the majority of the labial and lingual portions) or any other simple or complex shape desired, whether now known or hereafter discovered.

In a preferred method of the instant invention, annual protrusion 25 and circular protrusion 45 are incorporated into the CAD-CAM design stage for core 20. In one embodiment core 20 is milled from a ceramic material. Suitable materials for core 20 include, but are not limited to, Lava™ two-stage zirconium dioxide system offered by 3M ESPE, and the Precident™ one-stage Bio-HIP Y-TZP (High Heat and Isostatic Pressure formed ytrium stabilized tetragonal zirconium polymorph) offered by DCS of Switzerland. The Lava™ System utilizes a zirconia dioxide block that is CNC milled in a greenware state then secondarily heat sintered. The Precident System mills directly from the harder presintered Bio-HIP Y-TZP block.

Core 20, which includes protrusions 25 and 45, may be designed primarily manually using conventional CAD-CAM design software in which the user first designs core 20 without protrusions 25 and 45 in the manner in which core 20 of prior art substructures is design. The user then places pre-made shapes down over an image of core 20 on the screen, increases or decreases the existing shape, distorts the shape in one/multiple vanishing points, increases the volume of the shape from a point or angle, or free hand-paints an area to add protrusions 25 and 45. In a preferred embodiment however, the core 20 of the instant invention is designed primarily automatically by a CAD-CAM application that includes pull down annular shapes for protrusions 25 and 45 that are placed on/around the concentric image being made over the die for core 20 which is chosen from a library of tools/shapes in the software palate. Protrusions 25 and/or 45 may be enlarged (or reduced) in x, y, and z axis as necessary to bulk-up (or down) the core being designed. In such an automated CAD-CAM application, the CAD-CAM software recognizes (or identifies) the outer contour shape (i.e. the surface) of core 20 and conforms the inner surface of the pull-down annular shapes for protrusion 25 and 45 to the outer surface of core 20, such that the inner surface of protrusions 25 and 45 and the outer surface of core 20 are aligned. If desired, the outer surface of protrusions 25 and 45 can also follow the shape of the outer contour of core 20 by spacing each point of the outer surface of protrusion 25 an equal distance away from a corresponding point on the outer surface of core 20.

In another preferred embodiment of the instant invention core 20 of the instant invention is designed primarily automatically by a CAD-CAM application with the operator of the software pulling the structure from a point, line, cluster of points, etc., to distort a portion of the shape of the main body of the substructure without distorting the overall shape of the main body. Conventional modeling software primarily takes a shape and puts it in a “box” giving the operator the ability to pull at the corners to increase or decrease the volume of the shape or distort it. At all times, the operator is pulling the entire side of the 3D structure, not just the point. This is limiting as the software only allows symmetrical “pulls”, and has a geocentric pivot point for the shape within the box. In this embodiment of the instant invention, the modeling software utilizes a geocentric pivot point that may be displaced anywhere within the volume and/or along any line or curved line of the CAD-CAM image of core 20 so that the beam structures of the instant invention (i.e. protrusions 25 and 45) may be “pulled” from the main body of the substructure (i.e. core 20) without otherwise distorting the shape of the image of the body from which it is pulled.

In yet another preferred embodiment, the beam structures of the instant invention are pre-designed into the main body of the substructure. In one such embodiment, a basic shape for the main body of the substructure is selected from a library of shapes available in the software application based upon the desired shape for the final restoration, with the reinforcement structure already built into the shape of the main body. Thus, the beam structures' shapes is selected simultaneously with the main body shape. The operator then pushes, pulls, takes away or otherwise erases portions of the pre-designed shape of the main body and the beam structures in the software to meet the needs for the specific restoration. Alternative embodiments of shapes that can be used as preliminary or starting shapes in this particular embodiment of the instant invention include “door-knob” shapes (as shown in U.S. Pat. No. 7,967,606) that can be selected from a library in the design software; and a “coke-bottle,” shape (as shown in U.S. Pat. No. 7,967,606). Once selected, the sides of the preliminary core shape that is selected may be morphed, distorted or otherwise modified by pulling, pushing, taking away, erasing, etc., from the contact points. In a preferred embodiment, the sides are morphed by pulling from the contact points in pre-planned arcs of differing diameters, and controlled by looking down from the top of the shape (i.e. along the y-axis) and/or form the side of the shape to determine where (i.e. how many degrees around the outer surface of the shape) the selected morphing will occur. Examples of morphing include but are not limited to dragging the sides of the original shape apically or occlusally along the y-axis to make a symmetrical or asymmetrical (such as a French curve) arch, dragging the sides along the x-axis outwardly from the body of the shape, and also along the z-axis. It will be appreciated that the blocks of material from which the substructure (such as core 20) is milled may be pre-manufactured in the pre-determined shapes discussed above to minimize the amount of waste material during the milling of the final substructure, once the substructure has been designed in accordance with the instant invention.

In still another preferred embodiment, the substructure (including the beam structures of the instant invention) is designed by first obtaining the desired shape for the restoration and then subtracting away or deconstructing from that shape to leave the desired substructure shape. In such an embodiment, the part of the final shape of the restoration that is subtracted is determined to maximize the aesthetic appearance of the final restoration by concealing the substructure. In one such embodiment, the software constructs a “mesh framework of point clusters” that are external to (or in addition to) the point clusters established by the scan of the original piece that is being restored (or scan of a model of the piece to establish the desired external appearance of the restoration). These point clusters are used to construct an image of a “concentric” substructure (concentric to the original piece) for the restoration. The operator then embellishes or diminishes certain key areas, after rendering of the substructure image, to design the final substructure shape. Once the basic, overall shape has been rendered, the computer then knows in 3d, through the point clusters, where the operator is working, allowing the operator to easily take away portions of the image to result in a final image for the substructure.

Although shown and described in connection with a crown implant, it will be appreciated that the reinforcement structure of the instant invention can be used in connection with any dental restorations, including crowns and/or bridges, and including implant and/or restorations supported by teeth. Further, it will be appreciated that the materials used to manufacture the substructure (as well as the veneer) of the instant invention are not limited to those described herein. Although the inventive substructure is particular well suited for use with substructures manufactured of zirconium and other comparable ceramics, the inventive support structure may be utilized in connection with substructures manufactured from any other suitable material without departing from the spirit and scope of this instant invention.

It will also be appreciated that although the preferred method of the instant invention utilizes CAD-CAM design software, other methods of design (such as free-hand design, hologram or virtual reality modeling) now known or hereafter developed can be utilized without departing from the spirit and scope of the instant invention. Further, it will be appreciated that the inventive reinforcement may be used in connection any manufacturing process for crowns or bridges now known or hereafter discovered, including but not limited to simultaneous milling of a core and implant abutment, milling the core and abutment as a single piece, or milling of crowns and bridges from blocks or rods, etc. In addition, it will be appreciated that the infrastructures of the instant invention may be manufactured in methods other than the milling discussed herein. Alternative methods include but are not limited to press, lay-up, green ware production and subsequent milling or hand finishing.

Referring to FIG. 4, a front elevation section view of a dental restoration substructure of an embodiment of the instant invention similar to those discussed above and with respect to FIGS. 2 and 3 is shown.

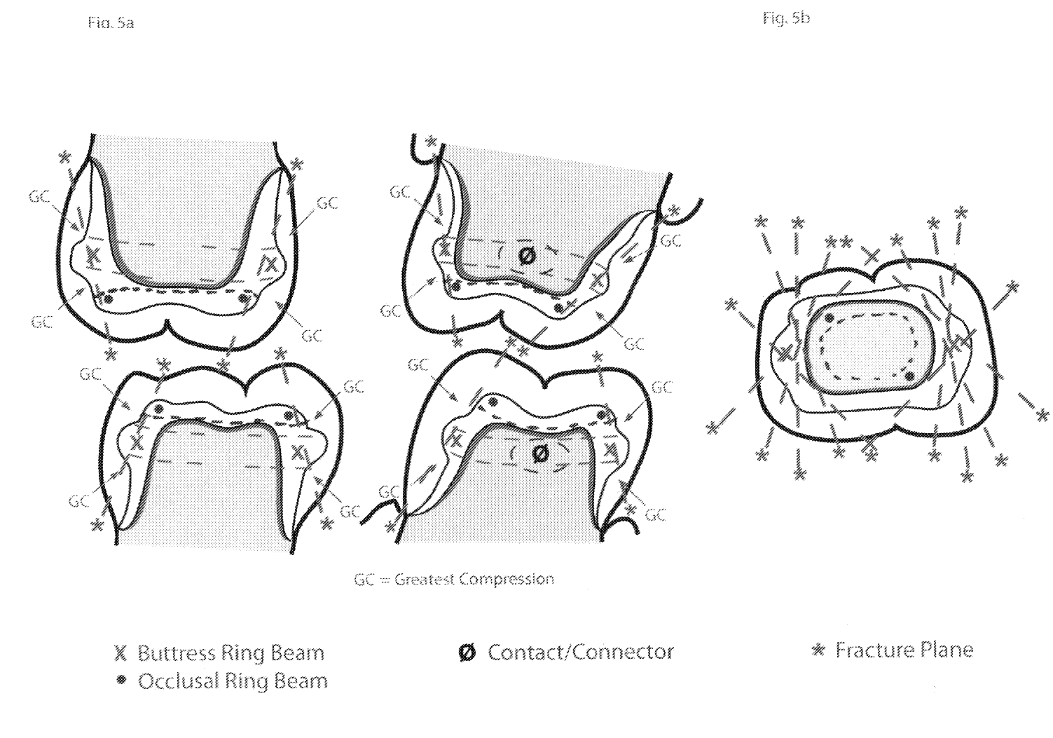

FIG. 5a showns a proximal section view of a dental restoration of another embodiment of the instant invention showing the areas of greatest compression of veneering porcelain. FIG. 5b is an occlusal section view of the dental restoration of FIG. 5a.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Although the foregoing detailed description of the present invention has been described by reference to an exemplary embodiment, and the best mode contemplated for carrying out the present invention has been shown and described, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall with in the true spirit and scope of the underlying principles disclosed and claimed herein. Consequently, the scope of the present invention is intended to be limited only by the attached claims, all matter contained in the above description and shown in the accompanying drawings shall be interpreted as illustrative and not in a limiting sense.

Having now described the features, discoveries and principles of the invention, the manner in which the invention is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

What is claimed is:

1. A dental restoration comprising:
an integrally formed structure body having an open distal end, a closed proximal-occlusal end and a side wall extending between said distal and proximal-occlusal ends, said structure body including a support structure protruding from an outer surface of a wall of said structure body, wherein said structure body forms a hollow framework for coping over a dental preparation;
a veneer formed over and contacting said outer surface of said wall and said support structure; and
said support structure located generally proximate to a location of stress and/or strain and protruding a distance from said surface of said wall, purposefully providing support for said veneer and purposefully reducing and/or distributing sheer stress that is borne by said structure body, said veneer and/or the dental restoration.

2. The restoration as claimed in claim 1 wherein a shape of said structure body is determined through use of a software application.

3. The restoration as claimed in claim 2 wherein the software application is a CAD-CAM application.

4. The restoration as claimed in claim 2 wherein the software application determines a shape of said support structure by identifying an outer contour shape for said structure body, and conforming generally at least a portion of said support structure shape to said outer contour shape of said structure body and said dental restoration.

5. The restoration as claimed in claim 4 wherein said identifying and conforming are accomplished generally automatically by a software application.

6. The restoration as claimed in claim 2 wherein the software application determines a shape of said support structure generally simultaneously with determining a structure body outer contour shape.

7. The restoration as claimed in claim 2 wherein the software application determines a shape of said structure body by subtracting from a final desired shape for said dental restoration.

8. The restoration as claimed in claim 1 wherein said restoration is designed to be manufactured through a material take-away manufacturing methodology.

9. The restoration as claimed in claim 1 wherein said restoration is designed to be manufactured through a material take-away manufacturing methodology.

10. The restoration as claimed in claim 1 wherein said restoration is designed to be manufactured through a material build-up manufacturing methodology.

11. The restoration as claimed in claim 1 wherein said restoration is designed to be made of successive layers of veneering material.

12. The restoration as claimed in claim 1 wherein said restoration is designed to be formed as one of successive components.

13. The restoration as claimed in claim 1 wherein said veneer comprises a coating.

14. The restoration as claimed in claim 1 wherein said support structure includes an annular support structure protruding from said outer surface of said side wall at a location spaced from, but generally near, said proximal-occlusal end of said structure body.

15. The restoration as claimed in claim 1 wherein said support structure has a smooth unifying shape and generally encircles an entire periphery of said structure body.

16. The restoration as claimed in claim 1 wherein said closed proximal-occlusal end includes a generally circular support protrusion that generally resides below said proximal-occlusal ends and marginal ridges of said structure body.

17. A dental restoration comprising:
an integrally formed structure body having an open distal end, a closed proximal-occlusal end and a side wall extending between said distal and proximal-occlusal ends, said structure body including an annular support structure protruding from said outer surface of said side wall at a location spaced from, but generally near, said proximal-occlusal end of said structure body, said support structure having a unifying shape and generally encircling an entire periphery of said structure body, wherein said structure body forms a hollow framework for a coping over a dental preparation, said closed proximal-occlusal end including a generally circular support protrusion that generally resides below said proximal-occlusal ends and marginal ridges of said structure body;
a veneer over and contacting said outer surface of said side wall, said support structure and said proximal-occlusal ends of said structure body; and
said support structure and said support protrusion located generally proximate to a location of stress and/or strain and protruding a distance from said surface of said side wall and occlusal wall surfaces and purposefully providing support for said veneer that purposefully reduces and/or distributes sheer stress that is borne by said structure body, said veneer and/or the dental restoration.

18. The restoration as claimed in claim 17 wherein a shape of said structure body is determined through the use of a software application.

19. The restoration as claimed in claim 18 wherein the software application is a CAD-CAM application.

20. The restoration as claimed in claim 18 wherein the software application determines a shape of said protruding support structure and/or said support protrusion by identifying an outer contour shape for said structure body, and conforming generally at least a portion of said protrusion shape to an outer contour shape of said structure body and said dental restoration.

21. The restoration as claimed in claim 20 wherein said identifying and conforming are accomplished generally automatically by the software application.

22. The restoration as claimed in claim 18 wherein the software application determines a shape of said protruding support structure and/or said support protrusion generally simultaneously with determining a structure body outer contour shape.

23. The restoration as claimed in claim 18 wherein the software application determines said shape of said structure body by subtracting from a final desired shape for said dental restoration.

24. The restoration as claimed in claim 17 wherein said restoration is designed to be manufactured through a material take-away manufacturing methodology.

25. The restoration as claimed in claim 17 wherein said restoration is designed to be manufactured through a material take-away manufacturing methodology.

26. The restoration as claimed in claim 17 wherein said restoration is designed to be manufactured through a material build-up manufacturing methodology.

27. The restoration as claimed in claim 17 wherein said restoration is designed to be made in successive layers of veneering material.

28. The restoration as claimed in claim 17 wherein said restoration is designed to be formed as one of successive components.

29. The restoration as claimed in claim 17 wherein said veneer comprises a coating.

30. The restoration as claimed in claim 17 wherein said unifying shape of said support structure is smooth.

31. A method of manufacturing a dental restoration comprising the steps of:
providing a structure body for the restoration that includes a support structure protruding from an outer surface of a side wall of the structure body at a location spaced from, but generally near, a proximal-occlusal end of the structure body;
veneering the structure body with an additional material having an ideal thermal expansion coefficient that is generally ≦1.0×10⁻⁶/° C. of the structure material to form a first modifier layer;
veneering the structure body with one or more successive layers of additional material;
treating the veneering layers at a temperature that is preferably slightly less than the recommended high firing temperature; and
withdrawing the structure from the treating step through a rapid cool-down protocol.

32. The method as claimed in claim 31 wherein the modifier layer is a special effect-shading that enhances an aesthetic appearance of the veneer.

33. The restoration as claimed in claim 1 wherein said structure body comprises a Zirconia material.

34. The restoration as claimed in claim 17 wherein said structure body comprises a Zirconia material.

35. The method as claimed in claim 31 wherein the structure body comprises a Zirconia material.
